(19) United States
(12) Patent Application Publication
(10) Pub. No.: US 2023/0248583 A1
(43) Pub. Date: Aug. 10, 2023

(54) DEVICES AND METHODS FOR PATCH DELIVERY

(71) Applicant: Boston Scientific Scimed, Inc., Maple Grove, MN (US)

(72) Inventors: Paul SMITH, Smithfield, RI (US); Shawn RYAN, Littleton, MA (US)

(73) Assignee: Boston Scientific Scimed, Inc., Maple Grove, MN (US)

(21) Appl. No.: 18/165,665

(22) Filed: Feb. 7, 2023

Related U.S. Application Data
(60) Provisional application No. 63/308,235, filed on Feb. 9, 2022.

(51) Int. Cl. A61F 13/26; A61F 13/20; A61F 2/966

(52) U.S. Cl. CPC A61F 13/266; A61F 13/2025; A61F 2/966; A61F 2013/2014

(57) ABSTRACT

A delivery system, including an inner member, an outer tube surrounding the inner member and movable axially relative to the inner member, wherein a space is defined between an outer surface of the inner member and an inner surface of the outer tube, an expandable element disposed in the space at a distal portion of the system, and a patch disposed in the space at the distal portion of the system, wherein the patch is radially outward of the expandable element.

### CROSS-REFERENCE TO RELATED APPLICATIONS

This application claims the benefit of priority under 35 U.S.C. § 119 from U.S. Provisional Application No. 63/308,235, filed Feb. 9, 2022, which is incorporated by reference herein in its entirety.

### TECHNICAL FIELD

This disclosure relates generally to medical devices and related methods of preparation and use thereof. More specifically, embodiments of this disclosure relate to patch delivery systems and related methods of using such systems to deliver a patch, among other aspects.

### BACKGROUND

Various endoscopic procedures are used for treatment of tissue. For example, an endoscopic procedure may be performed to remove large lesions (e.g., cancer), tunnel under the mucosal layer of the gastrointestinal (GI) tract, or treat tissues on other organs by passing outside of the GI tract. Other procedures include endoscopic full-thickness resection (EFTR) and endoscopic treatment of post-surgical issues, such as perforation, post-surgical leaks, breakdown of surgical staple lines, or GI bleeding within a lumen of the body.

These procedures may increase the risk of perforating the wall of the GI tract or may require closure of the GI tract wall as part of the procedure. Current patch or prophylactic materials may require pressure to be applied or a fluidic to provide moisture or activators to the patch. Systems for delivery of such patches are desirable.

### SUMMARY OF THE DISCLOSURE

According to an example, a delivery system may comprise an inner member, an outer tube surrounding the inner member and movable axially relative to the inner member, wherein a space may be defined between an outer surface of the inner member and an inner surface of the outer tube, an expandable element disposed in the space at a distal portion of the system, and a patch disposed in the space at the distal portion of the system, wherein the patch may be radially outward of the expandable element.

In another example, the expandable element may be a self-expanding stent or scaffold. A proximal end of the expandable element may be fixedly attached to the inner member. The expandable element may be in a collapsed configuration, and wherein a first portion of the expandable element may overlap with a second portion of the expandable element. The patch may be in a folded configuration, and a portion of the patch may be radially inward of the first portion of the expandable element and radially outward of the second portion of the expandable element. The patch may include an adhesive on a radially outermost surface of the patch. A liner may be positioned between an inner surface of the patch and an outer surface of the expandable element to inhibit the patch from adhering to the expandable element. A length of the patch may be less than a length of the expandable element. A proximal most end of the stent may be proximal to a proximal most end of the patch. A gap may be between two free ends of the patch, wherein the gap may extend along the length of the patch. The delivery system may further comprise a fluid lumen having an opening at a proximal end of the system and in fluid communication with the space, wherein the fluid lumen may extend into an opening at a proximal portion of a radially outer surface of a handle at a proximal end of the outer tube. A proximal end of the fluid lumen may include a port for connection to a fluid source. Moving the outer tube proximally relative to the inner member may expose an entirety of the patch and at least a portion of the expandable member, so that the portion of the expandable member expands and applies a radial force to the patch. At least a portion of the expandable member may be uncoated. The expandable member may be asymmetric for contacting less than a full circumference of a bodily lumen.

According to an example, a delivery system may comprise an inner member, an outer tube surrounding the inner member and movable axially relative to the inner member, wherein an annular space may be defined between an outer surface of the inner member and an inner surface of the outer tube, a self-expanding stent disposed in the annular space at a distal portion of the system, a patch disposed in the annular space at the distal portion of the system, wherein the patch may be radially outward of the stent, and a fluid lumen having an opening at a proximal end of the system and in fluid communication with the space, wherein the patch may include an adhesive on a radially outer surface of the patch and a non-adhesive on a radially inner surface of the patch in contact with the stent, wherein the length of the patch may be less than the length of the stent, wherein a proximal most end of the stent may be proximal to a proximal most end of the patch, wherein a gap may be formed between two free ends of the patch, wherein the gap may extend along the length of the patch, and wherein moving the outer tube proximally relative to the inner member may expose an entirety of the patch and at least a portion of the stent, so that the portion of the stent expands and applies a radial force to the patch. At least a portion of the stent may be uncoated. A width of the patch may be less than an inner circumference of the outer tube.

According to an example, a method of delivering a patch to a target tissue site may comprise positioning a delivery system adjacent to the target tissue site, wherein the delivery system may include an outer tube surrounding an inner member, wherein a space may be defined between an outer surface of the inner member and an inner surface of the outer tube, a stent is disposed in the space at a distal portion of the system, a patch is disposed in the space at the distal portion of the system, and the patch is radially outward of the stent, moving the outer tube proximally relative to the inner member exposing the patch and at least a portion of the stent, wherein the portion of the stent may apply a radial force to the patch to apply the patch to the target tissue site, moving the outer tube distally relative to the inner member to retrieve the portion of the stent into the space, and withdrawing the delivery system from the target tissue site and leaving the patch adhered to the target tissue site. A method of delivering a patch to a target tissue site may further comprise delivering fluid from a proximal portion of the system to a distal portion of the system via a fluid lumen to apply fluid between the outer tube and the inner member, to the stent and the patch, before proximal movement of the outer tube relative to the inner member.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is a perspective view of an exemplary patch delivery system, according to aspects of this disclosure.

FIG. 2 is a cross-sectional view of a distal portion of an exemplary patch delivery system, according to aspects of this disclosure.

FIG. 3 is a cross-sectional view of a distal portion of an exemplary patch delivery system, according to aspects of this disclosure.

FIG. 4 is a side partial cross-sectional view of a distal portion of an exemplary patch delivery system in a deployed state, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the patient. Throughout the figures included in this application, arrows labeled “P” and “D” are used to show the proximal and distal directions in the figure. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

Embodiments of this disclosure relate to systems and methods for delivering a patch to a treatment site by applying a radial force to the patch. In addition, such delivery systems and methods may deliver fluid to the patch during delivery to activate the patch and adhere the patch to the treatment site.

Although the term endoscope may be used herein, it will be appreciated that other devices, including, but not limited to, duodenoscopes, colonoscopes, ureteroscopes, bronchoscopes, laparoscopes, sheaths, catheters, or any other suitable delivery device or medical device may be used in connection with the devices of this disclosure.

The patch may be a biodegradable and/or biocompatible patch of any suitable shape and any suitable dimension, e.g. based on the nature of the target tissue site. The patch may have any shape such as, e.g., square, rectangular, oval, circular, among other possible shapes. In some examples, the thickness of the patch may be on the order of millimeters, e.g. ranging from about 0.7 mm to 2.0 mm. Moreover, the patch may be of any suitable material, e.g., polysaccharides (chitosan, cellulose, starch, alginates, etc.) that may be further modified with synthetic biocompatible materials (PGA, PLA, PCA, PEG, etc.).

The patch may be substantially dry during delivery to a target site. In some examples herein, the patch may be moistened during delivery to the target tissue site or as the patch is being applied to the target tissue site. For example, the patch may be exposed to fluid via a lumen of the delivery system and/or through contact with bodily fluids inside the body. According to some aspects of this disclosure, moistening the patch may assist in its application to the tissue target site.

In some examples herein, the patch comprises an adhesive, e.g., to assist in applying the patch to tissue and/or maintaining the patch in place after application, and/or to assist in delivery of the patch to the target site. In some examples herein, the patch may comprise an adhesive on only one side, e.g. the side of the patch facing the tissue target site. Adhesives suitable for this disclosure may be natural, e.g., comprising a natural polymer or derived from a natural polymer, or synthetic. Exemplary adhesives include, but are not limited to, gelatin (including, e.g., thrombin/gelatin), fibrin (e.g., fibrin glue), cyanoacrylate, polyethylene glycol (PEG), and albumin (including, e.g., albumin glutaraldehyde). The adhesive may be at least partially resorbable.

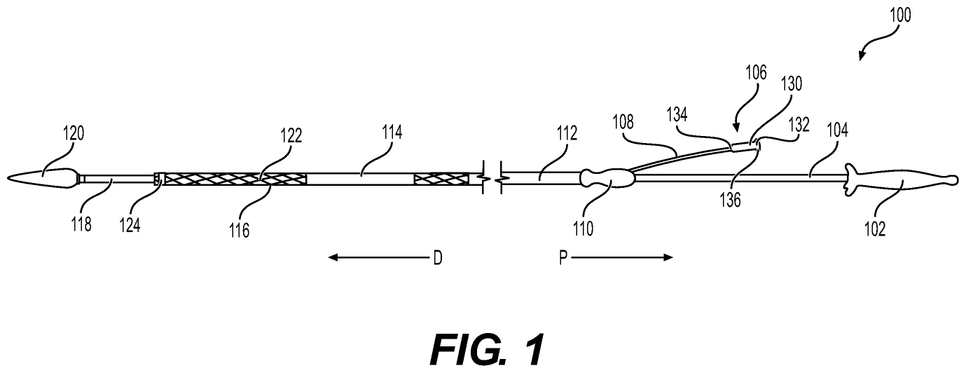

FIG. 1 is a perspective view of an exemplary patch delivery system. A proximal portion of a patch delivery system 100 may include an inner tubular member 104 and an outer tubular member 112. A handle 102 is disposed at a proximal end of the inner tubular member 104 and is fixedly attached to the inner tubular member 104. A handle 110 is disposed at a proximal end of the outer tubular member 112 and is fixedly attached to the outer tubular member 112. The handle 110 may include an inner lumen to receive a portion of the inner tubular member 104 and to facilitate axial movement of the inner tubular member 104 within the outer tubular member 112. The handle 110 may include an opening at a proximal portion of a radially outer surface of the handle 110 configured to receive a lumen 108.

The lumen 108 may be coupled to a port 106 disposed at a proximal end of the lumen 108. The lumen 108 and the port 106 may extend radially outward relative to handle 110 and in the proximal direction so as to form an angle less than 90 degrees with respect to a longitudinal axis of the patch delivery system 100 (and particularly of tubular members 104, 112). The port 106 may include a cylindrical, hollow body 130 with a first opening 132 at its proximal end and a second opening 134 at its distal end. A rim 136 may surround the opening 132. The second opening 134 leads into the lumen 108. The openings 132 and 134 of the port 106 may facilitate fluid flow from an outside fluid source to the lumen 108, through a lumen of the outer tubular member 112, and to a distal portion of the patch delivery system 100. In another example, the diameter of the opening 132 may be larger than the diameter of the opening 134. In another example, the diameter of the body 130 of the port 106 may be larger than the diameter of the lumen 108. In another example, the port 106 and the lumen 108 may be movable in the proximal and/or distal directions. For example, the port 106 may move in the proximal and/or distal directions, but may not move beyond an opening at a proximal portion of a radially outer surface of the handle 110. The lumen 108 may move in the proximal and/or distal directions beyond an opening at a proximal portion of a radially outer surface of the handle 110. In embodiments, the lumen 108 may extend into the handle 110, and a distal end of the lumen 108 may couple to the outer tubular member 112 in a manner permitting fluid to flow from within lumen 108 to within outer tubular member 112. For example, the lumen 108 may terminate in the handle 110, and a terminal, proximal end of outer tubular member 112 may be fluidly coupled with a distal end of the lumen 108 via any suitable connection means. Alternatively, the lumen 108 may extend the length of the patch delivery system 100 and terminate proximate a distal portion of the patch delivery system 100, at or near patch 114.

The lumen 108 may be used to apply fluid between elements of the patch delivery system 100 (e.g., a restraining tube 116, an inner member 118, a stent 122) at the distal portion of the patch delivery system 100 and the patch 114 as the elements and the patch 114 are being delivered to a target tissue site. The lumen 108, or an additional lumen not shown) may be used to apply fluid directly onto the patch 114 as the patch 114 is being applied to the target tissue site.

The restraining tube 116 is disposed at the distal portion of the patch delivery system 100 and may be a part of or coupled to the outer tubular member 112. For example, the outer tubular member 112 may extend to the distal portion of the patch delivery system 100 and may be coupled to, or otherwise integral with, the restraining tube 116. The restraining tube 116 may include a marker band 124 at or just proximal to its distal end for viewing via, for example, fluoroscopy or other imaging modality. The restraining tube 116 may have a length sufficient to extend over the stent 122 and the patch 114. The restraining tube 116 may be flexible and transparent.

The distal portion of the patch delivery system 100 may further include an inner member 118 that may be a part of or coupled to the inner tubular member 104. For example, the inner tubular member 104 may extend to the distal portion of the patch delivery system 100 and may be coupled to, or otherwise integral with, the inner member 118. The inner member 118 is within a lumen of, and movable axially, relative to the restraining tube 116. In embodiments, the inner member 118 may be tubular with a channel extending longitudinally therein, and in other embodiments, the inner member 118 may be a solid shaft without such a channel.

Relative axial movement of the handles 102 and 110 permits axial movement of the inner tubular member 104 and the inner member 118 relative to outer tubular member 112 and the restraining tube 116, respectively. For example, the handle 102 may be axially moved between distal and proximal positions, as the handle 110 remains stationary, so as to axially move the inner tubular member 104 and the inner member 118 axially. Such movement may be done while keeping the handle 110 fixed or relatively fixed to allow independent or substantially independent movement of the inner tubular member 104 and the inner member 118, while the outer tubular member 112 and the restraining tube 116 remain fixed or relatively fixed. Similarly, the handle 110 may be axially moved between distal and proximal positions, as the handle 102 remains stationary, so as to axially move the outer tubular member 112 and the restraining tube 116. Such movement may be done while keeping the handle 102 fixed or relatively fixed to allow independent or substantially independent movement of the outer tubular member 112 and the restraining tube 116, while the inner tubular member 104 and the inner member 118 remain fixed or relatively fixed. In another example, the handles 102 and 110 may be moved or manipulated in concert as a pair, so that the tubular members 104, 116 move with the outer tubular member 112 and the restraining tube 116.

An expandable element, such as the stent 122, is disposed at the distal portion of the patch delivery system 100. The expandable element can be an alternative structure configured to apply a radial force to a patch, such as a balloon. The stent 122 may be self-expandable and made of a shape memory material, such as nitinol. The self-expandable property of the stent 122 may allow for the stent 122 to apply pressure to the patch 114 during application of the patch 114 to a target tissue site. The stent 122 may be made of braided wires, for example. The stent 122 may be coated, uncoated, or have portions coated and uncoated. Uncoated portions of the stent 122, leaving openings between adjacent wires, will allow fluid from the lumen 108 to pass through the stent 122 and reach the patch 114, to moisten the patch 114 for improved adherence to tissue.

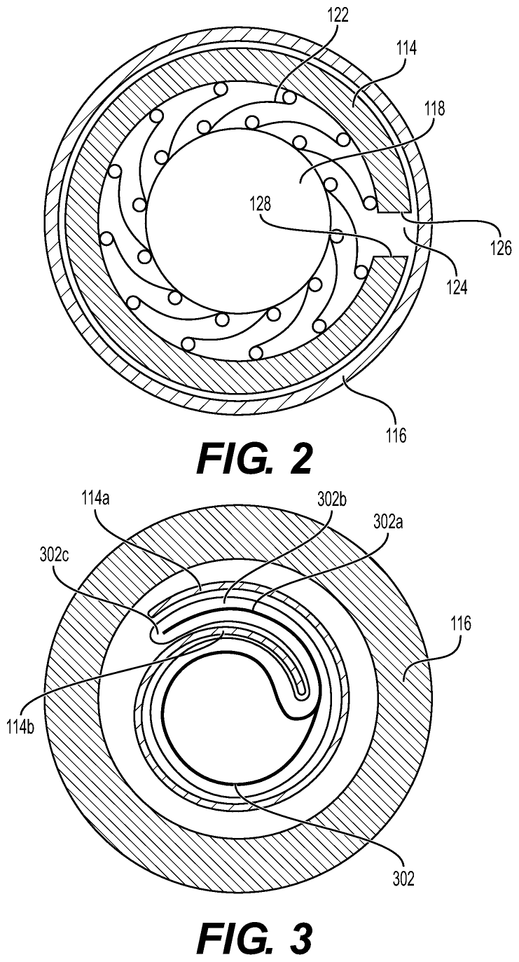

Before delivery and application of the patch 114 to a target tissue site, the stent 122 is disposed on an outer surface of the inner member 118 and held in a restrained configuration (i.e., the stent 122 has a diameter less than the diameter of the stent 122 in its expanded, resting configuration) by the restraining tube 116 (as shown in FIG. 2). The stent 122 and the restraining tube 116 may form an annular space for the patch 114 to be disposed within. In other examples, the annular space formed by the stent 122 and the restraining tube 116 can be of varying sizes to accommodate patches of various thicknesses as described above.

Further shown in FIG. 2, the patch 114 is disposed between the stent 122 and the restraining tube 116 in a folded, or curled, configuration. In some examples, the patch 114 may fully cover the stent 122 in the expanded, resting configuration of the stent 122 and/or in the collapsed stent configuration. In some examples, the patch 114 may cover only a circumferential portion of the stent 122, in either the resting or collapsed configuration, especially in cases for treating less than a full circumference of a bodily lumen. The patch 114 may extend all or less than an entire length of the stent 122. FIG. 1 shows the patch 114 extending less than the length of the stent 122. FIG. 2 shows the patch 114 extending circumferentially around the stent 122 less than an entire circumference of the stent 122.

In some examples, the width of the patch 114, in an original, unfolded configuration, is related to the inner circumference of the restraining tube 116. In one example, the maximum width of the patch 114, in its original, unfolded configuration, is at most equal to the inner circumference of the restraining tube 116. In another example, the width of the patch 114, in its original, unfolded configuration, may be less than the inner circumference of the restraining tube 116, such that when the patch 114 is disposed between the stent 122 and the restraining tube 116 in its folded configuration, a gap 124 is formed between two free ends 126 and 128 of the patch 114. It will be appreciated that the size of the gap 124 may vary depending on the width of the patch 114. The gap 124 may extend along the entire length of the patch 114 in its folded configuration. The gap 124 may allow fluid to flow from a proximal portion of the patch 114 to a distal portion of the patch 114 and/or vice versa. This may allow for the entire patch 114 and/or a substantial portion of the patch 114 to be moistened during delivery and/or application to a target tissue site. The gap 124 also helps to ensure that portions of the patch 114 do not adhere together during delivery and implantation.

In another example, an outer surface of the patch 114 (i.e., the surface facing the target tissue site) may include any suitable adhesive material so as to assist with applying the patch 114 to the target tissue site. An inner surface of the patch 114 (i.e., the surface contacting the stent 122) may include any suitable non-adhesive material so as to prevent the patch 114 from adhering to the stent 122 during delivery and/or application of the patch 114 to the target tissue site. In another example, a liner made of any suitable non-adhesive material may be disposed between the stent 122 and the patch 114 to prevent the patch 114 from adhering to the stent 122 during delivery and/or application of the patch 114 to the target tissue site.

In another example, after assembly of the patch delivery system 100 using any of the features from any of the examples described above, the distal portion of the patch delivery system 100, along with the patch 114, may be navigated to a target tissue site of a bodily lumen 200. Navigation can be assisted by a guidewire (extending through the handle 102, the inner tubular member 104 and the inner member 118, and out of a distal tip 120). The distal tip 120 may have a distal opening for exit of a guidewire, may be flexible and atraumatic, and may be coupled to an end of the inner member 118. Navigation also may be assisted by an endoscope, or other scope, catheter or like device, having imaging, lighting, and other functionality typical of such devices. The patch delivery system 100 may be delivered through a working channel of such a scope or delivered “side saddle” along an outer portion of the scope, as the scope is navigated to the treatment site. During navigation and other portions of patch delivery, fluoroscopy may be used to visualize the marker band 124, and any other suitably placed marker bands, and assist in navigation and patch delivery.

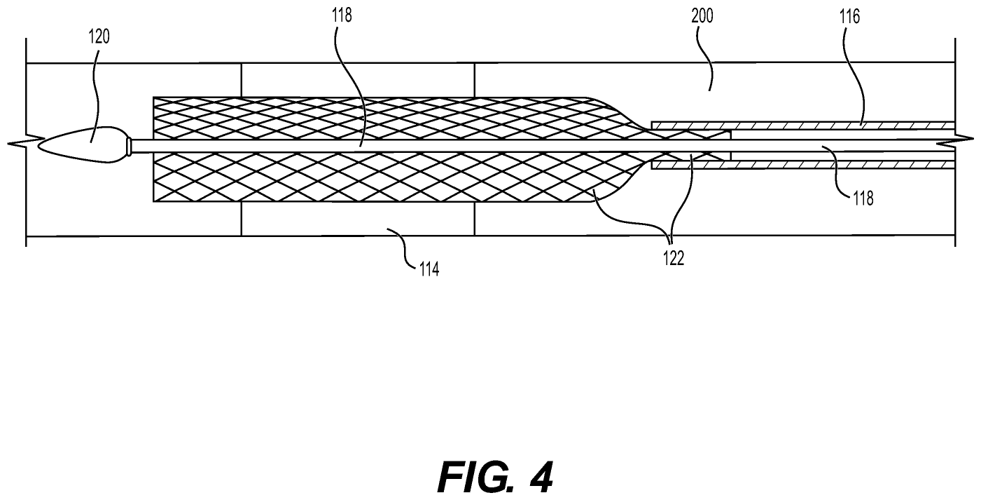

FIG. 4 is a side partial cross-sectional view of the distal portion of the patch delivery system 100 in a deployed state in the bodily lumen 200. Once the patch 114 and the stent 122 are aligned with, or otherwise adjacent, the target tissue site of the bodily lumen 200, the handle 110 from the proximal portion of the patch delivery system 100 may be moved proximally, relative to the handle 102. Correspondingly, the outer tubular member 112 and thus the restraining tube 116 move proximally and away from the distal tip 120 of the patch delivery system 100. As the restraining tube 116 moves proximally, a distal portion of the stent 122 transitions from its restrained configuration to an expanded configuration. Thus, as shown in FIG. 4, that distal portion of the stent 122, along with all of the patch 114 (to release the patch 114 from the patch delivery system 100), are exposed from the inner member 118 to contact an inner wall of the bodily lumen 200 including the target tissue site. Radial expansion of the distal portion of the stent 122 applies a radial pressure to the patch 114, which assists with the application of the patch 114 to the target tissue site. Contact of the patch 114 with bodily fluids inside the body may further assist with the application of the patch 114 to the target tissue site. The stent 122 may apply a radial pressure to the patch 114 until the patch 114 has sufficiently adhered to the target tissue site.

Once the patch 114 has been applied to the target tissue site, the handle 110 from the proximal portion of the patch delivery system 100 may be moved distally, relative to handle 102. Correspondingly, the outer tubular member 112 and thus the restraining tube 116 move distally to retrieve the portion of the stent 122 that was initially exposed and radially expanded. As the restraining tube 116 moves distally, the stent 122 transitions from its expanded configuration to its restrained configuration. The patch delivery system 100 may then be moved proximally and exit the bodily lumen 200, while leaving the patch 114 adhered to the target tissue site.

In another example, fluid may be introduced to the patch delivery system 100 using any outside fluid source, such as a syringe or fluid pump. An outside fluid source may provide fluid to the lumen 108 through the port 106, resulting in fluid flow from the lumen 108 to a distal portion of the patch delivery system 100. For example, fluid may flow from the lumen 108 to an annular space between the inner tubular member 104 and the outer tubular member 112 and ultimately in an annular space between the restraining tube 116 and the inner member 118. Fluid may also flow along the gap 124. Fluid may thereby contact the stent 122 and the patch 114 of the patch delivery system 100. In some examples, fluid may be provided as the patch delivery system 100 and the patch 114 are being delivered to the target tissue site. In another example, fluid may be sprayed onto the patch 114 as the patch 114 is being applied to the target tissue site, for example, using the same fluid transfer as described above. In another example, in an embodiment having the lumen 108 extend the length of the patch delivery system 100 to just proximal of the stent 122, and wherein the lumen 108 is independently movable, as radial pressure is applied to the patch 114 by the stent 122 as described above, the lumen 108 may then be moved distally towards the stent 122 and the patch 114. Once a distal end of the lumen 108 is positioned near the patch 114, fluid may be sprayed onto the patch 114. In one example, the stent 122 may be fully or partially uncoated so that lumen 108 may spray fluid directly onto patch 114 with minimal interference.

In embodiments, a proximal end of the stent 122 may be fixedly attached to an outer perimeter of one of the inner tubular member 104 and the inner member 118. This will ensure that the stent 122 is not completely released from a remainder of the patch delivery system 100 and irretrievable.

In another example, the patch delivery system 100 may include a scaffold structure 302 (shown in FIG. 3) instead of the stent 122 to assist with applying the patch 114 to a target tissue site. The scaffold structure 302 may be self-expandable and have any of the characteristics of the stent 122. The scaffold structure 302 may be folded upon itself in a collapsed configuration, so that a portion 302a of the scaffold 302 overlaps with another portion 302b of the scaffold 302. Scaffold portions 302a and 302b may be coupled together at their ends 302c. The patch 114 may be folded within the scaffold structure 302 in a folded, curled configuration. In this way, a portion 114a of the patch 114 is radially outside of another portion 114b of the patch 114, with scaffold portions 302a and 302b between patch portions 114a and 114b so that portions 114a and 114b do not adhere to one another. Use of the scaffold structure 302 may allow for a larger (e.g., increased width and/or thickness) patch 114 to be delivered to a target tissue site using the patch delivery system 100. In another example, the scaffold structure 302 may be asymmetric for contacting and/or treating less than a full circumference of a bodily lumen.

A proximal portion of the scaffold structure 302 may be coupled to a distal portion of the inner member 118 so that no portion of the inner member 118 extends through the scaffold structure 302 and the patch 114. In this example, the distal tip 120 may not be included. The scaffold structure 302 is held in its collapsed configuration and the patch 114 is held in its folded configuration by the restraining tube 116. Once the patch 114 and the scaffold structure 302 are aligned with the target tissue site, the handle 110 from the proximal portion of the patch delivery system 100 may be moved proximally. Correspondingly, the outer tubular member 112 and thus the restraining tube 116 move proximally, exposing the patch 114 and the scaffold structure 302. As the restraining tube 116 moves proximally, the scaffold structure 302 transitions from its collapsed configuration to an expanded configuration. In its expanded configuration, scaffold portion 302a may not overlie scaffold portion 302b, but remains coupled to scaffold portion 302b at ends 302c. Expansion of the scaffold structure 302 unfurls the patch 114 and applies pressure to the patch 114 until the patch 114 has sufficiently adhered to the target tissue site.

Once the patch 114 has been applied to the target tissue site, the handle 110 from the proximal portion of the patch delivery system 100 may be moved distally. Correspondingly, the outer tubular member 112 and thus the restraining tube 116 move distally and towards the target tissue site to retrieve the scaffold structure 302. As the restraining tube 116 moves distally, the scaffold structure 302 transitions from its expanded configuration to its collapsed configuration. The patch delivery system 100 may then be moved proximally and exit the bodily lumen, while leaving the patch 114 adhered to the target tissue site. Fluid may be applied to a distal portion of the delivery system 100 and/or sprayed directly onto patch 114 by the lumen 108 in a similar manner as described above.

In another example, a proximal portion of the scaffold structure 302 may be coupled to a distal portion of the inner member 118 and a distal portion of the scaffold structure 302 may be coupled to proximal portion of another inner member 118 extending to the distal tip 120. In another example, the inner member 118 may extend through the scaffold structure 302 and the patch 114 and to the distal tip 120.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

What is claimed is:

1. A delivery system, comprising:
an inner member;
an outer tube surrounding the inner member and movable axially relative to the inner member, wherein a space is defined between an outer surface of the inner member and an inner surface of the outer tube;
an expandable element disposed in the space at a distal portion of the system; and
a patch disposed in the space at the distal portion of the system, wherein the patch is radially outward of the expandable element.

2. The delivery system of claim 1, wherein the expandable element is a self-expanding stent or scaffold.

3. The delivery system of claim 1, wherein a proximal end of the expandable element is fixedly attached to the inner member.

4. The delivery system of claim 1, wherein the expandable element is in a collapsed configuration, and wherein a first portion of the expandable element overlaps with a second portion of the expandable element.

5. The delivery system of claim 4, wherein the patch is in a folded configuration, and a portion of the patch is radially inward of the first portion of the expandable element and radially outward of the second portion of the expandable element.

6. The delivery system of claim 1, wherein the patch includes an adhesive on a radially outermost surface of the patch.

7. The delivery system of claim 1, wherein a liner is positioned between an inner surface of the patch and an outer surface of the expandable element to inhibit the patch from adhering to the expandable element.

8. The delivery system of claim 1, wherein a length of the patch is less than a length of the expandable element.

9. The delivery system of claim 1, wherein a proximal most end of the stent is proximal to a proximal most end of the patch.

10. The delivery system of claim 1, wherein a gap is between two free ends of the patch, wherein the gap extends along the length of the patch.

11. The delivery system of claim 1, further comprising a fluid lumen having an opening at a proximal end of the system and in fluid communication with the space, wherein the fluid lumen extends into an opening at a proximal portion of a radially outer surface of a handle at a proximal end of the outer tube.

12. The delivery system of claim 11, wherein a proximal end of the fluid lumen includes a port for connection to a fluid source.

13. The delivery system of claim 1, wherein moving the outer tube proximally relative to the inner member exposes an entirety of the patch and at least a portion of the expandable member, so that the portion of the expandable member expands and applies a radial force to the patch.

14. The delivery system of claim 1, wherein at least a portion of the expandable member is uncoated.

15. The delivery system of claim 1, wherein the expandable member is asymmetric for contacting less than a full circumference of a bodily lumen.

16. A delivery system, comprising:
an inner member;
an outer tube surrounding the inner member and movable axially relative to the inner member, wherein an annular space is defined between an outer surface of the inner member and an inner surface of the outer tube;
a self-expanding stent disposed in the annular space at a distal portion of the system;
a patch disposed in the annular space at the distal portion of the system, wherein the patch is radially outward of the stent; and
a fluid lumen having an opening at a proximal end of the system and in fluid communication with the space;
wherein the patch includes an adhesive on a radially outer surface of the patch and a non-adhesive on a radially inner surface of the patch in contact with the stent;
wherein the length of the patch is less than the length of the stent;
wherein a proximal most end of the stent is proximal to a proximal most end of the patch;
wherein a gap is formed between two free ends of the patch, wherein the gap extends along the length of the patch; and
wherein moving the outer tube proximally relative to the inner member exposes an entirety of the patch and at least a portion of the stent, so that the portion of the stent expands and applies a radial force to the patch.

17. The delivery system of claim 16, wherein at least a portion of the stent is uncoated.

18. The delivery system of claim 16, wherein a width of the patch is less than an inner circumference of the outer tube.

19. A method of delivering a patch to a target tissue site, the method comprising:
positioning a delivery system adjacent to the target tissue site, wherein the delivery system includes an outer tube surrounding an inner member, wherein a space is defined between an outer surface of the inner member and an inner surface of the outer tube, a stent is disposed in the space at a distal portion of the system, a patch is disposed in the space at the distal portion of the system, and the patch is radially outward of the stent;
moving the outer tube proximally relative to the inner member exposing the patch and at least a portion of the stent, wherein the portion of the stent applies a radial force to the patch to apply the patch to the target tissue site;
moving the outer tube distally relative to the inner member to retrieve the portion of the stent into the space; and
withdrawing the delivery system from the target tissue site and leaving the patch adhered to the target tissue site.

20. The method of claim 19, further comprising delivering fluid from a proximal portion of the system to a distal portion of the system via a fluid lumen to apply fluid between the outer tube and the inner member, to the stent and the patch, before proximal movement of the outer tube relative to the inner member.
